(19) United States
(12) Patent Application Publication
(10) Pub. No.: US 2023/0073172 A1
(43) Pub. Date: Mar. 9, 2023

(54) PHOTOTHERAPY DEVICES AND METHODS

(71) Applicants: Zachary BOLDUC, Salisbury, MA (US); Roy Alan BOLDUC, Salisbury, MA (US); Douglas Price HEMLEPP, Newburyport, MA (US)

(72) Inventors: Zachary BOLDUC, Salisbury, MA (US); Roy Alan BOLDUC, Salisbury, MA (US); Douglas Price HEMLEPP, Newburyport, MA (US)

(21) Appl. No.: 17/801,495

(22) PCT Filed: Feb. 19, 2021
(86) PCT No.: PCT/US2021/018873

§ 371 Date: Aug. 22, 2022

Related U.S. Application Data
(60) Provisional application No. 62/995,927, filed on Feb. 21, 2020.
(60) Provisional application No. 63/107,515, filed on Oct. 30, 2020.
(60) Provisional application No. 63/109,015, filed on Nov. 3, 2020.
(60) Provisional application No. 63/109,058, filed on Nov. 3, 2020.

(51) Int. Cl. H01L 33/50; A61N 5/06

(52) U.S. Cl. CPC H01L 33/50; A61N 5/0616; A61N 5/062; A61N 2005/0656; A61N 2005/0659; A61N 2005/0661

(57) ABSTRACT

A product for use in treating a subject, incorporating a light-conversion medium which receives ambient light of broad spectrum, and emits light at one or more peak wavelengths or ranges of wavelengths (λ1, λ2, λ3) having greater intensity than corresponding intensity in the received broad spectrum ambient light. The products outlined are specifically designed for skin treatments of many different kinds, where the wavelengths chosen to have therapeutic or enhancing effects to the biological processes that occur inside or that directly affect the skin. The products can either exist as a unique treatment or be coupled with existing treatments in order to achieve an improved or differentiating result.

### CROSS REFERENCE TO RELATED APPLICATIONS

The foregoing application claims priority to U.S. Provisional Application No. 62/99,927, filed on Feb. 21, 2020; U.S. Provisional Application No. 63/109,058, filed on Nov. 3, 2020; U.S. Provisional Application No. 63/109,015, filed on Nov. 3, 2020, and U.S. Provisional Application No. 63/107,515, filed on Oct. 30, 2020, all of which are herein incorporated by reference as if provided herein in their respective entireties.

### FIELD OF THE INVENTION

The present invention is directed to apparatus, systems and methods for providing phototherapy treatments to a subject in need thereof.

### BACKGROUND OF THE INVENTION

Phototherapy, alternatively referred to as photo-bio-modulation (PBM) therapy involves the use of light sources, such as LED or laser light sources to treat certain medical conditions. For example, PBM therapy can use specific wavelengths, (such as red or infra-red wavelengths), and as shown in FIG. 1, to treat certain conditions, such as seasonal affectedness disorder and other aliments in humans.

At present, commercially available PBM technologies typically incorporate one or more electrically powered light sources, such as LEDs, to project specific light wavelengths onto the subject. For example, clinics and spas offering PBM typically use multiple light sources (shown in element 3 of FIG. 1), each one dedicated to a specific wavelength range, to direct therapeutic light on to a subject. While effective, such treatments require the subject to be stationary or be illuminated by artificial lights for a significant duration of time. Such approaches are both costly and time consuming for the subject.

Alternatively, there exists in the art a number of different approaches to deploy light therapy to patients without the use of electrically powered lights. For example, U.S. Pat. No. 9,295,855, herein incorporated by reference as if presented in its entirety, describes providing light therapy using one or more light spectrum conversion technologies that provide a light spectrum to the subject in the infrared or near infrared range. Likewise, U.S. Pat. No. 9,726,910, herein incorporated by reference as is presented in its entirety, describes using fluorescent dyes to provide phototherapy approaches.

However, while such disclosure rely on fluorescent dyes, the approaches described therein are costly, difficult to implement, and are not adapted to specific ailments or biological functions.

Thus, what is needed in the art are one or more approaches to providing PBM to a human subject using one or more passive light emission devices in a manner that is targeted to specific ailments, effective and cost efficient.

### SUMMARY OF THE INVENTION

In one or more implementations, the present invention is directed to one or more light conversion devices that receive and absorb broad spectrum ambient light, whether sunlight or indoor lighting, and emit light at one or more peak wavelengths, or ranges of wavelengths. In a particular implementation, the emitted wavelengths or wavelength ranges have greater intensity than the corresponding intensity of the wavelength in the received broad spectrum ambient light.

In one or more further particular implementations, one or phototherapy platforms are configured to deliver phototherapy to a subject. The phototherapy platform is configured with at least one emission compound configured to receive light across a broad spectrum and emit at least a portion of the total wavelength of the received broad spectrum, wherein the emitted portion of the wavelength is emitted at a greater intensity than the intensity of light within the corresponding portion of the broad spectrum absorbed.

In yet a further implementation, an emissive compound incorporated within the phototherapy platform and configured to emit light includes quantum dots, where the quantum dots are tuned to a specific emission spectra that mirrors the absorption spectra of Cytochrome C Oxidase or other transmembrane protein complexes that are involved in cellular respiration, repair or regulation.

In yet a further implementation, the phototherapy platform is incorporated into a lens, wherein the phototherapy platform is configured to emit light at an intensity that is suitable for regenerating cells and tissue of the eye.

In yet a further particular implementation, a quantum dot light source for phototherapy treatment is provided and described herein, wherein the light source is configured to emit light at the following wavelengths 590, 633, and 830 nm from available ambient light, thus enabling the user to receive an extended, prolonged period of treatment.

In yet a further implementation, the phototherapy platform described herein includes a lens comprising: a transparent substrate; and at least one excitation film layer disposed on the transparent substrate, the excitation film layer including a plurality of quantum dots, each quantum dot encapsulated in an encapsulation compound transparent to a first wavelength spectrum, wherein the quantum dot is configured to absorb ambient light within the first wavelength range and emit light within a second wavelength range, where the second wavelength range is encompassed by the first wavelength range.

In a further implementation, the excitation film layer is disposed between a semi-reflective layer and the subject in need of phototherapy, wherein the semi-reflective layer is configured to transmit light in the first wavelength spectrum through a first surface and reflect light within the second wavelength range from a second surface that is in contact with the excitation film layer.

In yet a further implementation, an emissive compound is configured to emit the absorbed light across a first wavelength range and emit light at a second wavelength range that is not within the first wavelength range.

In yet a further implementation, an emissive compound contains one or more quantum dots tuned to emit a specific emission spectrum that mirrors at least the absorption spectra for Cytochrome C Oxidase or other transmembrane protein complexes that are involved in cellular respiration, repair or regulation.

In yet a further implementation, the phototherapy platform is incorporated into a lens, wherein the phototherapy platform is configured to emit light at an intensity that is suitable for regenerating cells and tissue of the eye.

In yet a further particular implementation, a quantum dot light source for phototherapy treatment is provided and described herein, wherein the light source is configured to emit light at the following wavelengths 590, 633, and 830 nm from available ambient light, thus enabling the user to receive an extended, prolonged period of treatment.

In yet a further implementation, the phototherapy platform described herein includes a lens comprising: a transparent substrate; and at least one excitation film layer disposed on the transparent substrate, the excitation film layer including a plurality of quantum dots, each quantum dot encapsulated in an encapsulation compound transparent to a first wavelength spectrum, wherein the quantum dot is configured to absorb ambient light within the first wavelength range and emit light within a second wavelength range, where the second wavelength range is encompassed by the first wavelength range.

In a further implementation, the excitation film layer is disposed between a semi-reflective layer and the subject in need of phototherapy, wherein the semi-reflective layer is configured to transmit light in the first wavelength spectrum through a first surface and reflect light within the second wavelength range from a second surface that is in contact with the excitation film layer.

### BRIEF DESCRIPTION OF THE DRAWINGS

The subject matter disclosed herein is, in part, illustrated in the figures of the accompanying drawings which are meant to be exemplary and not limiting, in which like references are intended to refer to like or corresponding parts, and in which:

FIG. 1 represents phototherapy treatment in the treatment of target areas on humans in accordance with the prior art;

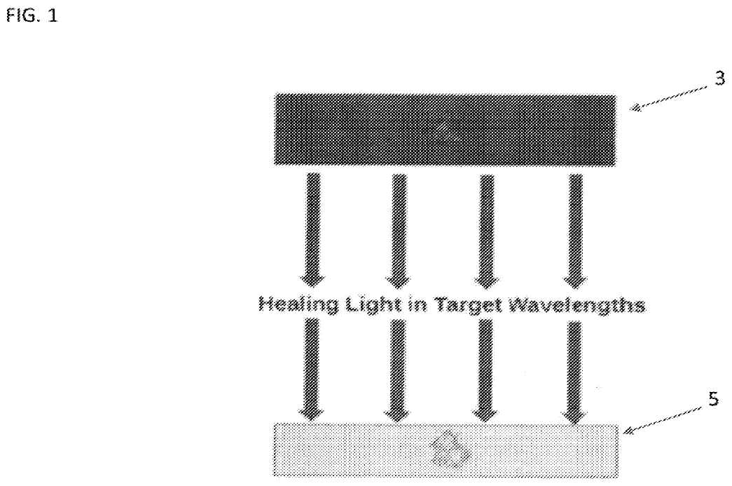

FIG. 2 illustrates the application of a product in accordance with one embodiment of the subject matter described in the treatment of a target area;

FIG. 3 illustrates in detail a product in accordance with one embodiment of the phototherapy platform;

FIG. 4 represents light conversion of ambient light of broad spectrum to light at one tuned wavelength emission gap having a defined wavelength range λn1-n2 and a greater intensity than corresponding intensity in the broad spectrum ambient light;

FIG. 5A illustrates a phototherapy platform in accordance with the phototherapy platform;

FIG. 5B illustrates an optical lens as one embodiment of a product in accordance with the present invention;

FIG. 6 illustrates a platform substrate according to one or more another embodiment of a product in accordance with the phototherapy platform;

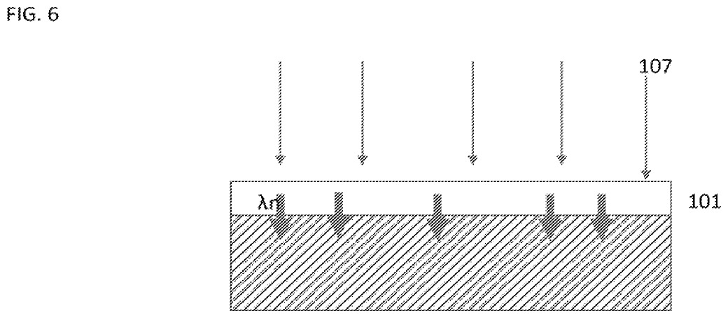

FIG. 7 illustrates an encapsulated fluid bandage as still another embodiment of a product in accordance with the phototherapy platform;

FIG. 8 illustrates an encapsulation element of still another embodiment of a product in accordance with the phototherapy platform;

FIG. 9A illustrates particular implementations of the phototherapy platform as described herein;

FIG. 9B illustrates particular implementations of the phototherapy platform as described herein;

FIG. 10 illustrates particular implementations of the phototherapy platform as described herein; and

FIG. 11 illustrates particular implementations in an alternative phototherapy platform as described herein.

### DETAILED DESCRIPTION OF CERTAIN EMBODIMENTS OF THE INVENTION

By way of overview and introduction, various embodiments of the apparatus, systems, compositions and methods described herein are directed towards a light phototherapy platform. In a particular implementation of the phototherapy platform described, the phototherapy platform is implemented as one or more\compositions designed to produce a given wavelength range of light when exposed to a broad spectrum of ambient light. In yet a further particular implementation, the phototherapy platform includes one or more films, filters, gels, creams and ointments containing emissive compounds that can be applied to the eyes, skin or other biological surface of a subject as part of an ailment treatment.

In a further particular implementation, the light phototherapy platforms include one or more encapsulated quantum dot formulations configured to emit light at a wavelength range suitable for use in phototherapy.

In a further particular implementation, a method is provided for delivering phototherapy to a subject using one or more encapsulated quantum dots. In a particular implementation, the phototherapy platform and methods of using the same provided herein are directed to providing therapy or otherwise providing phototherapy for eye, skin, wound and follicle treatments. For example, the wavelengths emitted by the phototherapy platform are useful for treatment rashes, scars, acne, wrinkles or other conditions of the skin. Likewise, the phototherapy platform described herein can be used to baldness, hair loss, thinning hair or other follicle treatments. By way of further example, the wavelengths emitted by the phototherapy platform are useful for treatment ocular conditions.

In one or more implementations, the foregoing provides one or more methods of providing a phototherapy platform to a person in need of treatment wherein the method includes the application of a fluid, gel or cream phototherapy platform as a treatment for one or more of the following ailments: rosacea, age related skin damage, eczema, acne, hair loss, psoriasis, and other skin ailments and diseases. For example, the phototherapy can be combined with one or more therapeutic compositions for treating a specific ailment such as hair loss. For instance, the phototherapy platform can be co-administered or co-formulated with at least one antihypertensive vasodilator compound, such as minoxidil.

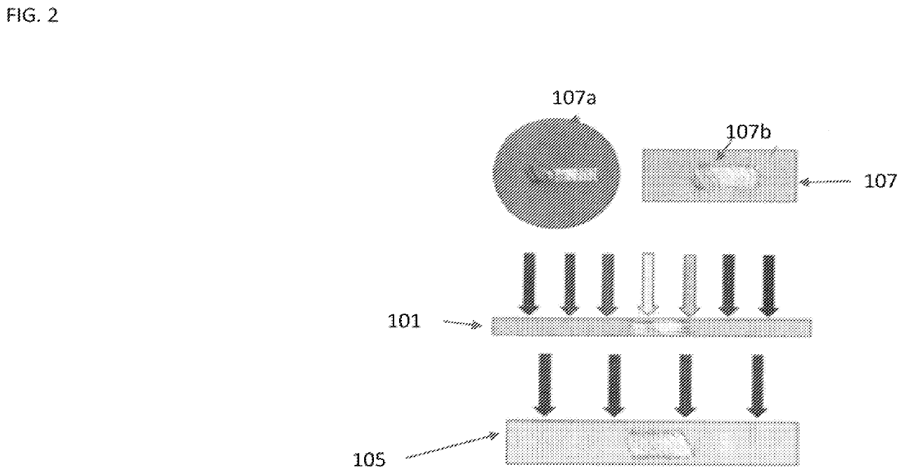

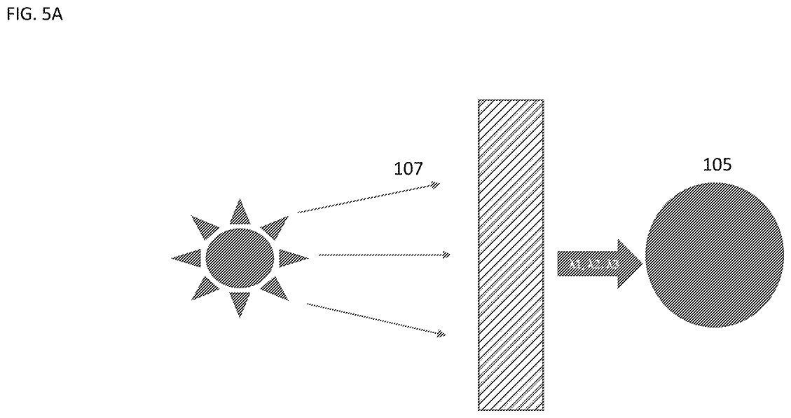

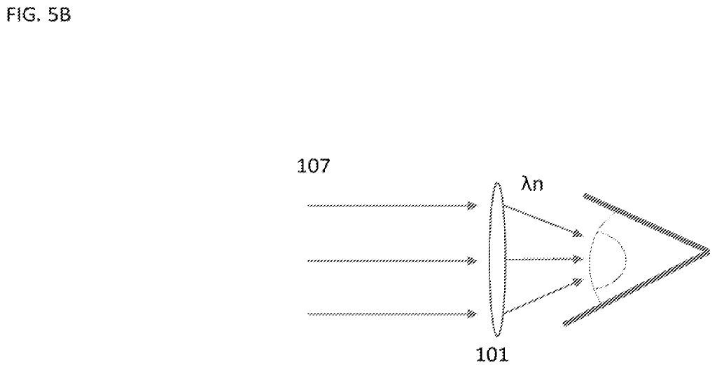

As provided in FIGS. 2, 5A and 5B, a phototherapy platform 101 in accordance with one embodiment of the subject matter described herein, is used in the treatment of a surface 105 using ambient light 107, in this embodiment ambient light can be sunlight 107a and/or indoor light 107b. Here, the surface 105 can be the skin or other biological surface of a mammal, such as a human. As shown in FIG. 5B, the phototherapy platform 101 can be incorporated into a lens or eye covering.

As described in more detail herein, and with particular reference to FIGS. 2-3 and 9A-9B, the phototherapy platform 101 includes one or more emission compounds 111 that are configured to emit light within in a pre-determined wavelength range when excited by ambient light. For example, the phototherapy platform is configured with one or more emission compounds 111 designed to absorb sunlight, artificial light or indirect light and emit light within pre-determined wavelength range.

For example, the phototherapy platform 101 includes one or more emissive compounds 111 configured to receive or absorb light across the visible spectrum and generate or emit light within the red and near-infrared wavelengths. In one particular implementation, the light emitted is configured for use in light therapy or other phototherapy applications.

In one or more further particular implementations, the intensity of the wavelength of light emitted by emissive compound 111 is higher than the intensity of the same wavelength of light absorbed by the emissive compound 111.

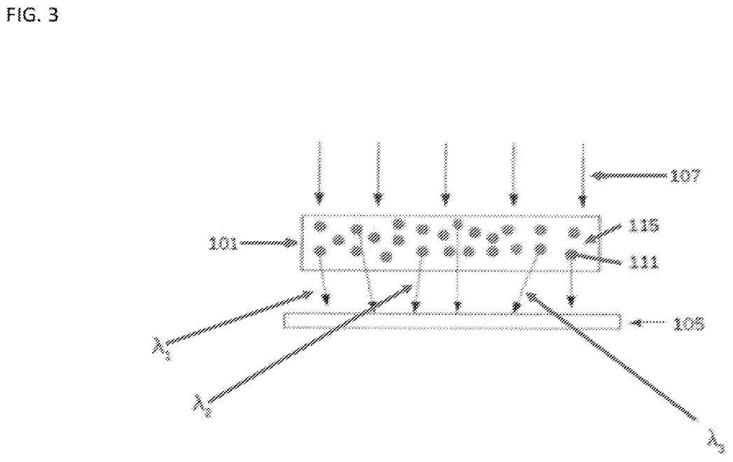

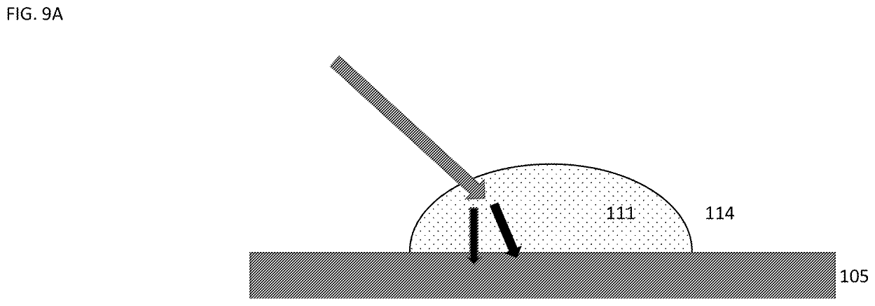

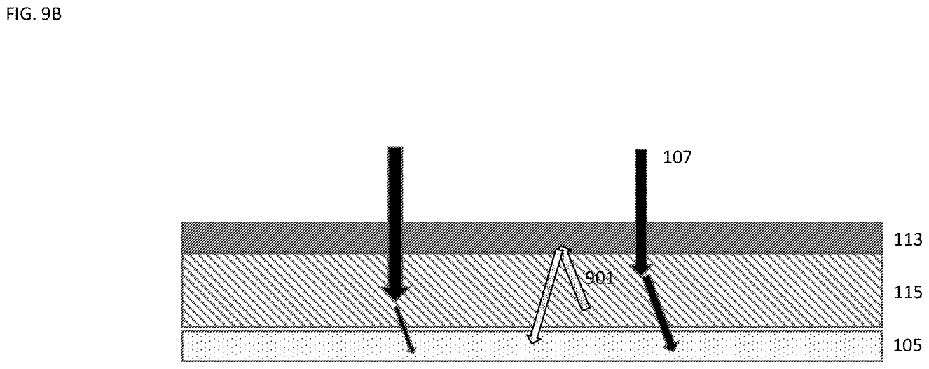

Continuing with FIGS. 3 and 9A-9B, and by way of non-limiting example, the emissive compound 111 is a nanocrystal or nanoparticle semiconductor. In one particular implementation, emissive compound 111 includes one or more quantum dots or Q-dots (QDs). It will be appreciated that QDs currently find application in display panels for color control and radio-medicine for improved radio-imaging. It will be further appreciated that when quantum dots are illuminated by an energy source, the quantum dot emits energy at a frequency that corresponds to the band gap of the semiconductor material used in the quantum dot, where the band gap is a function of the size of the nanocrystal. Examples of emissive compounds 111 include single shell QDs, multi-shell QDs, heavy metal QDs and/or non-heavy metal QDs. As noted, the emission properties of the QDs are dependent upon the size and shape of the nanoparticles or nanocrystals. Thus, different emission properties can be produced by varying or engineering the physical parameters of quantum dots, nanoparticles or nanocrystals.

In one embodiment the emissive compounds 111 includes silicon QDs. In one particular implementation, the emissive compounds 111 include lead, gold or silver QDs. Further exemplary materials suitable for use as quantum dots also include ZnS, ZnSe, ZnTe, CdS, CdSe, CdTe, GaN, GaP, GaAs, GaSb, InP, InAs, InSb, AlS, AlP, AlAs, AlSb, PbS, PbSe, Ge, and Si and ternary and quaternary mixtures thereof. The quantum dots may further include an overcoating layer of a semiconductor having a greater band gap. In one or more implementations, the emissive compound 111 are CuInS2/ZnS fluorescent nanocrystals.

In yet a further implementation, the emissive compounds 111 include one or more of elements selected from Group 14 elements. For example, the emissive compound 111 quantum dots are made from or incorporate one or more of the following elements carbon (C), silicon (Si), germanium (Ge), tin (Sn), lead (Pb), and flerovium (FI). In yet another configuration, the emissive compounds 111 include or incorporate nanodiamonds, diamond nanoparticles or other compound incorporating diamond particles that are less than 1 micrometer in size. In one or more further implementations, the emissive compound 111 includes carbon quantum dots that incorporate or include carbon particles having a size equal to or smaller than 10 nm. In a further implementation, the emissive compound 111 includes Perovskites compounds.

In certain configurations or implementations, the emissive compound 111 has a major fraction of particles having an average diameter of from about 0.1 nm to about 1000 nm, optionally from about 0.1 nm to about 100 nm, optionally from about 0.1 nm to about 10 nm, optionally from about 1 nm to about 10 nm, optionally from about 10 nm to about 100 nm.

By way of non-limiting implementation, the emissive compound 111 described herein is configured to receive incident or ambient light generated by one or more natural or artificial sources and emit light within the 500 to 800 nm wavelength range. In one or more particular implementations of the emissive compound 111 described herein is configured to emit light within the 590-850 nm wavelength ranges.

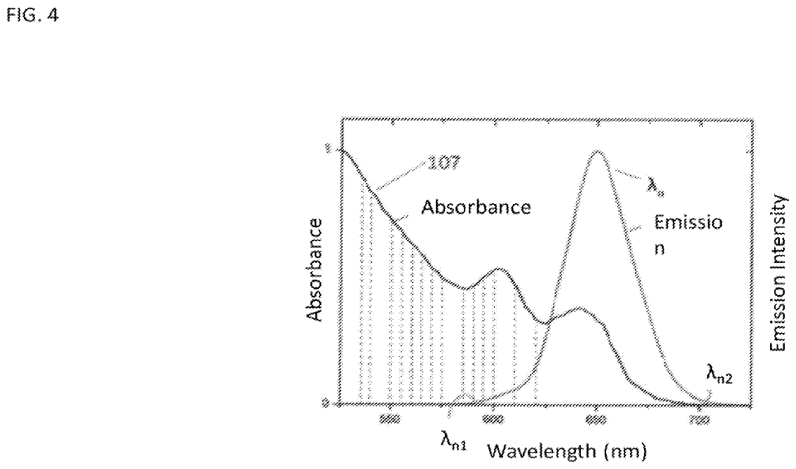

As shown in reference to FIGS. 4, 5A, and 5B, the emissive compound 111 converts broad spectrum ambient light 107 to light tuned to a specific wavelength emission gap having a defined wavelength range λn1-n2 and a greater intensity than corresponding intensity in the broad spectrum ambient light 107. For example, and in no way limiting, the phototherapy platform 101 is configured to absorb ambient light that includes light in a wavelength range of 500-600 nm. Here the absorbed light is at a first intensity level. The phototherapy platform is then configured to emit light within the narrower wavelength range at a second intensity. Here, the second intensity is greater than the first intensity, such that the phototherapy platform is configured to emit, without an external power source, a greater intensity of a specific wavelength across a narrower spectrum.

In one aspect, the emissive compound 111 emits light at one or more peak wavelengths or ranges of wavelengths (λ1, λ2, λ3) having greater intensity than corresponding intensity in the received broad spectrum ambient light.

In another embodiment the one or more peak wavelengths or ranges of wavelengths λ1, λ2, λ3 emitted by emissive compound 111 are from about 200 nm to about 3000 nm. In a further implementation, the light emitted by the emissive compound 111 includes light in the ultraviolet (UV) wavelengths, optionally from about 200 nm to about 400 nm, optionally in the UVA, optionally from about 310 nm to about 360 nm. In a further implementation, the emissive compound 111 emits light in the visible wavelength range, optionally from about 400 nm to about 700 nm. In a further implementation, the emissive compound 111 emits light in the infra-red (IR) wavelength range, optionally from about 700 nm to about 3000 nm. In a further implementation, the emissive compound 111 emits light optionally in the near infrared (NIR), optionally from about 810 nm to about 850 nm.

In one embodiment the one or more peak wavelengths or ranges of wavelengths λ1, λ2, λ3 are selected from about 200 nm to about 3000 nm. In one embodiment the one or more peak wavelengths or ranges of wavelengths λ1, λ2, λ3 are in the UV, optionally from about 200 nm to about 400 nm, optionally in the UVA, optionally from about 310 nm to about 360 nm. In one embodiment the one or more peak wavelengths or ranges of wavelengths λ1, λ2, λ3 are in the visible wavelength range, optionally from about 400 nm to about 700 nm. In one embodiment the one or more peak wavelengths or ranges of wavelengths λ1, λ2, λ3 are in the infrared wavelength range, optionally from about 700 nm to about 3000 nm, optionally in the near infrared, optionally from about 810 nm to about 850 nm.

In yet a further implementation, the emissive compound 111 is a QD with specific emission spectra that mirrors the absorption spectra of Cytochrome C Oxidase or other transmembrane protein complexes that are involved in cellular respiration, repair or regulation. In another implementation, the emissive compound 111 is a QD with specific emission spectra that mirrors the absorption spectra of one or more additional biological structures, such as but not limited to mitochondrial bound water as described in Sommer A P. Mitochondrial cytochrome c oxidase is not the primary acceptor for near infrared light-it is mitochondrial bound water: the principles of low-level light therapy. Ann Transl Med. 2019; 7(Suppl 1):S13. doi:10.21037/atm.2019.01.43, herein incorporated by reference in its respective entirety.

In one or more particular implementations, the emissive compounds 111 are configured to emit light that is then incident upon the surface 105 of the subject in the following wavelengths: 590, 633, and 830 nm. In one or more implementations of the phototherapy platform 101, the emissive compound 111 is configured to provide emitted light at the following wavelengths 570, 620, 680, 760, and 820 nm.

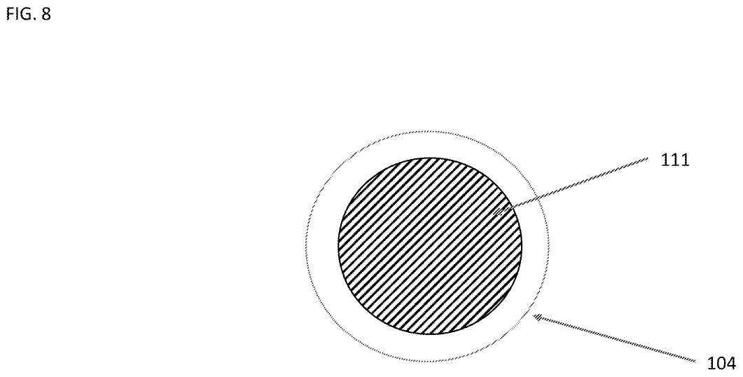

As further shown in FIG. 8, the emissive compound 111 is encapsulated in one or more encapsulation compounds 104. For example, and in no way limiting to the disclosures herein, the encapsulation compound 104 is a transparent compound that permits the transit of light across both the visible and infrared wavelength spectrums. In a further implementation the encapsulation compound 104 is configured to also be transparent to ambient light in the UV and near UV spectrum.

In one or more implementations the encapsulation compound 104 includes one or more three-dimensional, crosslinked polymer gel networks. In one or more further implementations, the encapsulation compound 104 includes or comprises a gel network. In one particular implementation, the gel network comprises poly (N,N-disubstituted acrylamide), polyacrylate esters, polyalkyl substituted vinyl ethers, and a gel network of polyglycol ethers or a mixture thereof.

In one or more implementations, the encapsulation compound 104 includes one or more degradable or non-degradable polymers, such as, but not limited to, synthetic polylactide/co-glycolide co-polymer, porous lauryllactame/caprolactame nylon co-polymer, hydroxyethylcellulose, polyelectrolyte monolayers, natural hydrogels (such as hyaluronic acid, gelatin and others or mixtures thereof).

In further or additional embodiments, the encapsulation compound 104 described herein is a hydrophilic or aliphatic coating (or mixture thereof), wherein the coating does not substantially adsorb to the skin of a mammalian subject. By way of non-limiting example, the encapsulation compound 104 is selected from polyethylene glycol, silica, silica-oxide, polyvinylpyrrolidone, polystyrene, polyquaternium(s), a protein or a peptide.

In a particular embodiment the encapsulated emissive compound 111 is incorporated into one or more biocompatible materials for direct application to the surface 105 of a subject. For example, and in no way limiting, the encapsulated emissive compound 111 is incorporated into a cream or gel composition that is appropriate for application to the tissue (such as but not limited to ocular tissues) or skin of a human subject. For example, instead of the emissive compounds 111 being individually encapsulated by an encapsulation compound 104, a plurality of emissive compounds 111 are encapsulated in an encapsulation compound 104 such as a gel formulation or a cream. In a further implementation, the encapsulation compound 104 that encapsulates a plurality of emissive compounds 111 selected is transparent to one or more wavelengths of light that will be absorbed by the encapsulated emissive compound 111. For instance, the encapsulation compound 104 is a cream or gel with suitable light transmissive properties.

By way of further example, the emissive compounds 111 are provided directly to the surface subject, such as for direct application to the skin or For example, the emissive compounds 111 are integrated into an encapsulation compound, which in turn is incorporated into a medicinal or therapeutic formulation, as eye drops, salves or ointments, In one particular implementation, the emissive compound 111 compound is (i) a bio-compatible material or intrinsically modified to be bio-compatible or (ii) encapsulated so as to prevent direct contact with the subject.

One or more alternative implementations, as shown in FIGS. 3, and 7-11, the emissive compound 111 is embedded in a light-transmissive carrier or support medium 115 capable of being formed, machined or processed. In another implementation, the emissive compound 111 is first encapsulated into an encapsulation compound 104 and then embedded in the support medium 115. In one particular implementation the support medium 115 is a thin film, flexible film, rigid film or semi-rigid film. In a further implementation, the emissive compound 111 and/or encapsulation compound 104 are incorporated into a transparent, translucent, or opaque support medium 115.

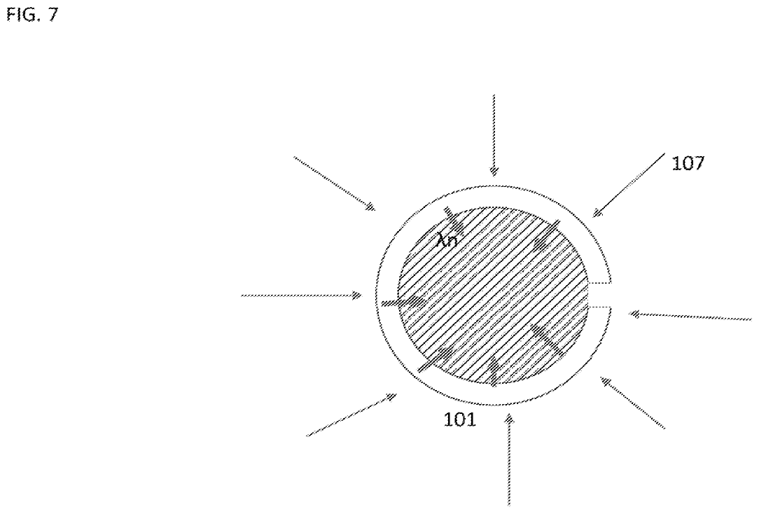

In another embodiment the support medium 115 itself is a fluid material, such as a gel, a cream, or a liquid. In one embodiment, as illustrated in FIG. 7, the phototherapy platform 101 that incorporates a fluid support medium 115 provides a composition which can be applied directly to a user, such as for application to the skin, for example, as a topical cream, gel or ointment.

In another embodiment the support medium 115 is a fluid. In yet a further implementation, a fluid support medium 115 is encapsulated so as to prevent direct contact with the user. In this embodiment, the encapsulated fluid support medium 115 can have the form of a fluid bandage, as illustrated in FIGS. 9A, 10-11.

In one or more implementations, the emissive compound 111 is incorporated into an adhesive bandage. For example, the support medium 115 is a film or thin material strip that can be applied to the surface of a subject. For example, the support medium can be implemented as a thin film strip that adheres to the surface 105 of a subject, such as to the subject's skin. In one embodiment the phototherapy platform 101 is another form skin dressing configured to be placed on the skin of a subject such that the subject is able to receive phototherapy from the dressing. In one embodiment the phototherapy platform 101 includes a support medium fashioned or machined into eyeglasses, goggles, visors, monocles or contact lenses.

For example, as shown in FIG. 5B, the phototherapy platform 101 is an eyeglass, monocle, goggle, visor or other product configured to be fitted over the eyes. For example, the phototherapy platform 101 is any device that is used in the treatment of an ocular condition, such as a retinal condition. In another embodiment the product is a disposable or non-disposable contact lens. It will be appreciated that any corrective or therapeutic eyewear that is suitable for ameliorating or correcting an ocular condition can incorporate the phototherapy platform 101 as provided herein. For example, the phototherapy platform 101 is a contact lens (as shown in FIG. 5) that incorporates the emissive compound 111 embedded in encapsulation compound 104. In one particular implementation, the phototherapy platform 101 receives ambient light 107 of broad spectrum, (such as but not limited to ultraviolet (UV), infra-red (IR) and visible light). This ambient light is absorbed by the emissive compound 111. The emissive compound 111 then emits light at one or more peak wavelengths (Δn) or ranges of wavelengths (such as λ1, λ2, λ3) having greater intensity than corresponding intensity in the received broad spectrum ambient light.

In one particular implementation, the phototherapy platform 101 is configured with one or more filtering layers that are configured to permit the passage of light within specific wavelength ranges. For example, the phototherapy platform 101 is equipped with one or more filters that prohibits certain wavelengths from reaching the surface 105 of the subject. For example, a filter layer is disposed between the support medium 115 and the surface 105 such that light not having a specific wavelength (such as IR wavelengths) are prevented from striking the surface 105.

In another embodiment the support medium 115 is a fluid. In yet a further implementation, a fluid support medium 115 is encapsulated so as to prevent direct contact with the user. In this embodiment, the encapsulated fluid support medium 115 can have the form of a fluid bandage. For example, the arrangement disclosed herein and illustrated in FIGS. 9A,10-11 can be incorporated into a fluid bandage.

In one or more implementations, the emissive compound 111 is incorporated into an adhesive bandage. For example, the support medium 115 is a film or thin material strip that can be applied to the surface of a subject. For example, the support medium can be implemented as a thin film strip that adheres to the surface 105 of a subject, such as to the subject's skin. In one embodiment the phototherapy platform 101 is another form of wound or skin dressing configured to be placed on the skin of a subject such that the subject is able to receive phototherapy from the dressing.

In another implementation, the emissive compound 111 is incorporated into a support medium 115 configured as a fluid bandage, as shown in FIGS. 7-8. For example, a fluid bandage could incorporate one or more emissive compounds 111, each turned to emit a particular wavelength range in response to being illuminated by ambient light. In one particular implementation, the fluid bandage includes one or more polymers configured to adhere to the skin of a human. For instance, the polymer is selected from a group that includes one or more of polyvinylpyrrolidone, ethyl cellulose, pyroxylin/nitrocellulose, poly(methylacrylate-isobutene-monoisopropylmaleate), and acrylate or siloxane polymers (hexamethyldisiloxane or isooctane solvent based). In a further implementation, the liquid bandage also includes one or more antimicrobial agents.

Turning now to FIG. 9A, the phototherapy platform is configured to direct the light emitted by the emissive compound 11 to the skin of the subject. For example, the emissive compound 111 and encapsulation compound 104 are encased or encapsulated in a support medium 115 that secures the encapsulated emissive compounds 102 on a surface 105. As noted, the support medium 115 is itself can be encapsulated in a barrier or other device or element that retains the support medium 115 where the support medium 115 is a fluid support medium.

Here, a barrier 114 encapsulates or otherwise encloses a given quantity of encapsulated emissive compounds 111. In one particular implementation, the barrier 114 is formed of a transparent material that permits a broad spectrum of light to pass therethrough. In a particular implementation, the barrier 114 is formed of one or more transparent or semi-transparent materials that are configured to permit the passage of light of particular wavelengths.

In an alternative configuration, as shown in FIG. 9B, the phototherapy platform 101 includes one or more anti-reflective layers provided as a coating or component to a lens. By way of non-limiting example, the phototherapy platform 101 includes one or more emissive compounds 111 that are configured to convert light within the visible light spectrum to light within the NIR or IR spectrum. However, it will be appreciated that the conversion process is not 100% efficient such that all of the converted light is directed to the surface 105. It is possible that light within a given wavelength range emitted by the emissive compound 111 will be emitted in a direction opposite that of the surface 105. Thus, in one or more implementations the phototherapy platform 101 is configured with one or more reflective coatings and reflective layers 113. In one arrangement, the emissive compound 111 (integrated in a support medium 115) is disposed between the reflective layer 113 and the surface 105 of the subject. In this arrangement, the emissive compound 111 is configured to emit light in a wavelength that is reflected by the reflective layer 113. In this arrangement, light (shown in black arrows) is either absorbed by the emissive compound 102 and emitted at a suitable phototherapy wavelength in an orientation that allows the light to reach the surface 105 of the subject. However, where light emitted by the emissive compound 111 is oriented such that it would travel along a path that does not intersect with the surface 105 (as shown in the dashed arrow), the reflective layer 113 reflects that light (now in the converted wavelength suitable for phototherapy) back to the surface 105 of the subject.

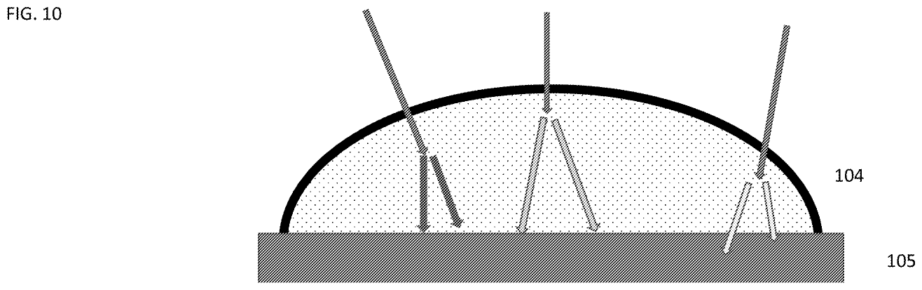

As shown in FIG. 10, multiple emissive compounds 111 are provided within an encapsulation compound 104 or support medium 115. For example, the encapsulated emissive compound 111 are integrated into a support medium 115. Here, emissive compounds 111 having different emission wavelengths are provided within the same support medium 115 such that upon absorption of the same broad-spectrum ambient light, the different emissive compounds 111 emit different wavelengths (as shown in the different colored arrows). In one particular implementation, the support medium 115 incorporates a plurality of emissive compounds 111 where at least a portion of the emissive compounds 111 are configured to emit light at the following wavelengths: 590, 633, and 830 nm.

In a further particular implementation, the different emissive compounds 111 are configured to produce wavelength peaks about the following wavelengths 590, 633, and 830 nm. In a further implementation, the emissive compounds 111 are configured to produce wavelengths substantially within +2%, 5%, 10% of 590, 633, and 830 nm respectively. In a further implementation, the emission intensity outside this range of the emissive compounds 111 is less than 5% of the highest emission intensity within the emitted range.

In a particular implementation, the different emissive compounds 111 are configured to produce wavelength peaks directed to different biological functions. For example, in one particular implementation, one or more emissive compounds 111 are directed to emit light in a wavelength that is tuned to disrupt viral replication or the growth of bacterial colonies. For example, one or more emissive compounds 111 are configured to emit light between 220 and 280 nm. An additional one or more emissive compounds 111 are configured to emit light in different wavelength ranges to promote the alleviation of one or more skin ailments. healing. In yet a further implementation, the emissive compound 102 includes QDs with specific emission that mirrors the absorption spectra of Cytochrome C Oxidase or other transmembrane protein complexes that are involved in cellular respiration, repair or regulation.

Thus, the two different wavelengths are configured to work synergistically in a skin ailment treatment package. In a further implementation, the number of emissive compounds 111 incorporated into a given fluid support medium 115 such that the overall intensity of the light generated is below a given threshold. For example, the amount of UV light emitting emissive compound 111 is such that the resulting emission of UV light is below a threshold intensity that would cause cellular damage in humans. In another arrangement, the threshold intensity is configured to be below the intensity harmful to humans by above the minimum amount needed to disrupt biological functions of microorganisms.

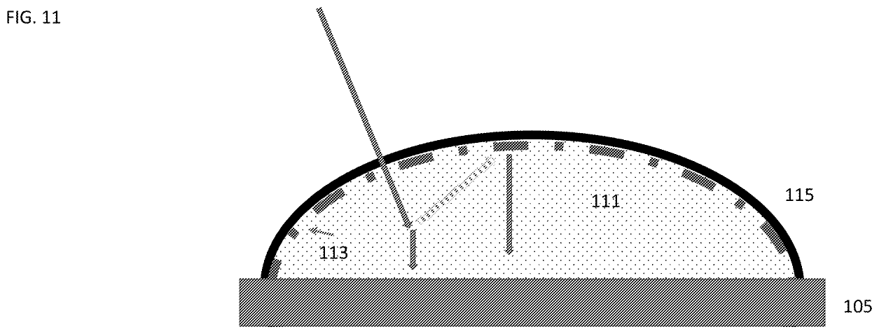

In an alternative configuration, as shown in FIG. 11, the phototherapy platform 101 is utilizes one or more anti-reflective layers. By way of non-limiting example, the phototherapy platform 101 includes one or more emissive compounds 111 that are configured to convert light within the visible light spectrum to light within the NIR or IR spectrum. However, it will be appreciated that the conversion process is not 100% efficient such that all of the converted light is directed to the surface 105. It is possible that light within a given wavelength range emitted by the emissive compound 111 will be emitted in a direction opposite that of the surface 105. Thus, in one or more implementations, the phototherapy platform 101 is configured with one or more reflective coatings and reflective layers 113. In one arrangement, the emissive compound 111 (integrated in a support medium 115) is disposed between the reflective layer 113 and the surface 105 of the subject. In this arrangement, the emissive compound 111 is configured to emit light in a wavelength that is reflected by the reflective layer 113. Light (shown in black arrows) is absorbed by the emissive compound 102 and emitted at a suitable phototherapy wavelength in an orientation that allows the light to reach the surface 105 of the subject. However, where light that is emitted by the emissive compound 111 is oriented such that it would travel along a path that does not intersect with the surface 105, the reflective layer 113 reflects that light (now in the converted wavelength suitable for phototherapy) back to the surface 105 of the subject or for reabsorption and emission by a emissive compound 111.

In a further implementation of the phototherapy platform 101 provided herein, the emissive compound 111 comprises a particulate material. In one embodiment the particulate material is transparent, translucent, or opaque. In one embodiment the particulate emissive compound 111 is suspended in the support medium 115. In one embodiment the particulate emissive compound 111 is bonded to the support medium 115, forming a hybrid material. In one embodiment the support medium 115 a solid material, such as a plastics material, a polymeric material, semi-conductor material, or crystalline material, optionally the solid support medium is rigid, semi-rigid or flexible. In one embodiment the product is a gel used to treat skin ailments. In another embodiment the phototherapy platform 101 is paired with existing skin treatment materials and active ingredients to enhance the overall performance of said existing treatment.

In one particular implementation, a method is provided to prevent or reduce scar tissue formation. Typically, scarring can consist of one or more of the following: raised hypertrophic scars or fibrosis, depressed atrophic scars, hyperpigmentation, hyperpigmentary redness or telangectasia. In a particular implementation the emissive compound 111 is embodied in a phototherapy platform 101 designed to eliminate or minimize hypertrophic scars (which are composed of an excess of collagen). For example, the emissive compound 111 is configured to emit the light that is tuned to enhance the production of collagen dissolving enzymes (metalloproteinases) such as MMP-1 (collagenase) in the skin or tissue of a human. In one or more implementations, the emissive compound 111 is paired with other compounds designed or configured to enhance the reduction of scar tissue in the skin of a human.

In one or more particular implementations, the phototherapy platform 101 is incorporated into a product for use in or application to living subjects, including, humans and animals. Such uses can include:

isolated light therapy;

scar treatment;

skin treatment, for example, in the treatment of acne, eczema, rosacea, or pigment loss;

hair loss;

anti-wrinkle treatment;

anti-aging/collagen treatment;

pain relief, for example, rheumatoid arthritis;

protection against UVA and UVB.

In one or more implementations, the product incorporating the phototherapy agents are prepared for topical administration, e.g. as an ointment, a gel, a drop, a patch or a cream. For topical administration to body surfaces using, for example, creams, gels, drops, ointments and the like, the compounds of the present disclosure can be prepared and applied in a physiologically acceptable diluent with or without a pharmaceutical carrier. Adjuvants for topical or gel base forms may include, for example, sodium carboxymethylcellulose, polyacrylates, polyoxyethylene-polyoxypropylene-block polymers, polyethylene glycol, wood wax alcohols, isostearic acid, cetyl alcohol, stearyl alcohol, white petrolatum, polysorbate 60, sorbitan monostearate, glycerin, xanthan gum, water, benzyl alcohol, methylparaben, and propylparaben. Additional additives may be selected from the group consisting of waxes, soaps, sorbitan esters, fatty acids, fatty acid esters, fatty acid oils, borates, cresol, chlorocresol, cellulose, methylcellulose, hydroxypropylcellulose, acacia, and the like.

The disclosure further contemplates a method of light therapy treatment of a subject in need thereof, such method comprising generating a modified light spectrum output using a photoluminescent light spectrum conversion device of the present disclosure and exposing a body region of the subject.

In one or more further implementations, A light spectrum conversion device is provided for absorbing and converting sunlight or other available ambient light to a modified light spectrum output that contains 50% or more of its output radiant in the 625-675 nm spectral range, wherein the light spectrum conversion device includes one or more quantum dots, configured to absorb 50% of light incident upon the spectrum conversion device in the 500-600 nm range.

In one or more particular embodiments of the subject matter described herein, a particular implementation includes a lens comprising: a transparent substrate; and at least one excitation film layer disposed on the transparent substrate, the excitation film layer includes a plurality of quantum dots, each quantum dot encapsulated in an encapsulation compound transparent to a first wavelength spectrum, wherein the quantum dot is configured to absorb ambient light within the first wavelength range and emit light within a second wavelength range, where the second wavelength range is encompassed by the first wavelength range.

The lens of any previous implementation provided herein, wherein the first wavelength range includes at least the UV and visible wavelengths and the second wavelength includes the near infrared and infrared wavelength.

The lens of any previous implementation provided herein, wherein the intensity of the second wavelength emitted by the encapsulated quantum dot is suitable for regenerating cells and tissue of the eye.

In one or more particular embodiments of the subject matter described herein, a particular implementation includes a product for use in treating a subject, incorporating a emissive compound which receives ambient light of broad spectrum, and emits light at one or more peak wavelengths or ranges of wavelengths (λ1, λ2, λ3) having greater intensity than corresponding intensity in the received broad spectrum ambient light.

In a further arrangement, the product of described in any previous implementation, wherein the one or more peak wavelengths or ranges of wavelengths λ1, λ2, λ3 are from about 200 nm to about 3000 nm, optionally in the UV, optionally from about 200 nm to about 400 nm, optionally in the UVA, optionally from about 310 nm to about 360 nm, optionally in the visible, optionally from about 400 nm to about 700 nm, optionally in the IR, optionally from about 700 nm to about 3000 nm, optionally in the NIR, optionally from about 810 nm to about 850 nm.

In a further arrangement, the product of described in any previous implementation, wherein the emissive compound comprises a particulate material.

In a further arrangement, the product of described in any previous implementation, wherein the particulate emissive compound is supported by a light-transmissive carrier or support medium, optionally the support medium is transparent, translucent, or opaque.

In a further arrangement, the product of described in any previous implementation, wherein the particulate emissive compound is suspended in the support medium.

In a further arrangement, the product of described in any previous implementation, wherein the support medium is a solid material, such as a plastics material, a polymeric material, or a glass, optionally the solid support medium is rigid, semi-rigid or flexible.

In a further arrangement, the product of described in any previous implementation, wherein the product is an eye glass, which is used in the treatment of an ocular condition.

In a further arrangement, the product of described in any previous implementation, wherein the product is a contact lens, which is used in the treatment of an ocular condition, such as a retinal condition.

In a further arrangement, the product of described in any previous implementation, wherein the support medium is a fluid material, such as a gel, a cream, or a liquid.

In a further arrangement, the product of described in any previous implementation, wherein the product is a composition which can be applied directly to the subject, such as for application to ocular tissues, for example, as a topical cream or ointment, or to the eyes, for example, as drops, optionally the emissive compound is (i) a bio-compatible material or intrinsically modified to be bio-compatible or (ii) encapsulated so as to prevent direct contact with the subject, optionally the encapsulated fluid support medium has the form of a fluid bandage.

In a further arrangement, the product of described in any previous implementation, wherein the support medium is a film.

In a further arrangement, the product of described in any previous implementation, wherein the product is a dressing, which is used, for example, in the treatment of an ocular conditions.

In one or more particular embodiments of the subject matter described herein, a particular implementation includes an emissive compound has a major fraction of particles having an average diameter of from about 0.1 nm to about 1000 nm, optionally from about 0.1 nm to about 100 nm, optionally from about 0.1 nm to about 10 nm, optionally from about 1 nm to about 10 nm, optionally from about 10 nm to about 100 nm.

In a further arrangement of the emissive compounds described herein, the emissive compound comprises a nanocrystal or nanoparticle semiconductor.

In a further arrangement of the emissive compounds described herein, wherein the emissive compound comprises quantum dots or Q-dots (QDs).

In a further arrangement of the emissive compounds described herein, wherein the emissive compound includes single shell QDs, multi-shell QDs, heavy metal QDs and/or non-heavy metal QDs.

In a further arrangement of any of the products described herein, wherein the product is used in: isolated light therapy; ocular conditions, for example, retinal conditions, such as diabetic radiotherapy, and macular degeneration; protection against UVA and UVB.

In one or more particular embodiments of the subject matter described herein, a particular implementation includes a composition comprising: a plurality of quantum dots, each quantum dot encapsulated in an encapsulation compound transparent to a first wavelength spectrum, wherein the quantum dot is configured to absorb ambient light within the first wavelength range and emit light within a second wavelength range, where the second wavelength range is encompassed by the first wavelength range, wherein a first portion of the plurality of quantum dots are configured to emit light within the second wavelength range that is different from the light emitted by a second portion of the plurality of quantum dots.

In one or more particular embodiments of the subject matter described herein, a particular implementation includes a composition for use in treating a skin ailment, deficiency or non-aesthetic symptom, incorporating a light-conversion medium which receives ambient light of broad spectrum, and emits light at one or more peak wavelengths or ranges of wavelengths (λ1, λ2, λ3) having greater intensity than corresponding intensity in the received broad spectrum ambient light.

In one or more further implementations of the products described herein, wherein the one or more peak wavelengths or ranges of wavelengths λ1, λ2, λ3 are from about 200 nm to about 3000 nm, optionally in the UV, optionally from about 200 nm to about 400 nm, optionally in the UVA, optionally from about 310 nm to about 360 nm, optionally in the visible, optionally from about 400 nm to about 600 nm, optionally in the IR, optionally from about 600 nm to about 3000 nm, optionally in the NIR, optionally from about 810 nm to about 850 nm.

In one or more further implementations of any products described herein, wherein the light-conversion medium comprises a carrier medium and a light conversion element.

In one or more further implementations of any of the products described herein, wherein the particulate light-conversion medium is supported by a light-transmissive carrier or support medium, optionally the support medium is transparent, translucent, or opaque.

In one or more further implementations of any of the products described herein, wherein the particulate light-conversion medium is suspended in the support medium or molecularly bonded to the support medium, thus creating a new hybrid material.

In one or more further implementations of any of the products described herein, wherein the support medium is a solid material, such as a plastics material, a polymeric material, or an organic material, optionally the solid support medium is rigid, semi-rigid or flexible.

In one or more further implementations of any of the products described herein, wherein the support medium is a fluid material, such as a gel, a cream, or a liquid.

In one or more further implementations of any of the products described herein, wherein the fluid material includes one or more compounds includes one or more collagen dissolving enzymes.

In one or more further implementations of any of the products described herein, wherein the fluid material can be used as an additive to an adhesive or liquid bandage.

In one or more further implementations of any of the products described herein, wherein the product is a composition which can be applied directly to the subject, such as for application to the skin, for example, as a topical cream, gel, or ointment, (i) a bio-compatible material or intrinsically modified to be bio-compatible or (ii) encapsulated so as to prevent direct contact with the subject, optionally the encapsulated fluid support medium has the form of a gel, cream, or ointment for topical application to the skin of a human.

In one or more further implementations of any of the products described herein, wherein the support medium is a film.

In one or more further implementations of any of the products described herein, wherein the skin ailment, deficiency or non-aesthetic symptom is scar tissue and wounds.

In one or more further implementations of any of the products described herein, wherein the product is a gel, cream, or ointment which is used, for example, in the treatment of a skin condition.

In one or more further implementations of any of the products described herein, wherein the product can be coupled or mixed in with one or more additional therapeutic compounds, components, ingredients or methods in order to enhance or differentiate the existing product.

In one or more further implementations of any of the products described herein, wherein the light-conversion medium has a major fraction of particles having an average diameter of from about 0.1 nm to about 1000 nm, optionally from about 0.1 nm to about 100 nm, optionally from about 0.1 nm to about 10 nm, optionally from about 1 nm to about 10 nm, optionally from about 10 nm to about 100 nm.

In one or more further implementations of any of the products described herein, wherein the light-conversion medium comprises a nanocrystal or nanoparticle semiconductor.

In one or more further implementations of any of the products described herein, wherein the light-conversion medium comprises quantum dots or Q-dots (QDs).

In one or more further implementations of any of the products described herein, wherein the light-conversion medium includes single shell QDs, multi-shell QDs, heavy metal QDs and/or non-heavy metal QDs.

In one or more further implementations of any of the products described herein, wherein the light-conversion medium includes silicon quantum dots which are molecularly bonded to the transparent medium.

In one or more further implementations of any of the products described herein, wherein the product is used in a liquid bandage incorporating one or more antimicrobial agent.

While this specification contains many specific embodiment details, these should not be construed as limitations on the scope of any embodiment or of what can be claimed, but rather as descriptions of features that can be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Particular embodiments of the subject matter described in this specification have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results.

Publications and references to known registered marks representing various systems cited throughout this application are incorporated by reference herein. Citation of any above publications or documents is not intended as an admission that any of the foregoing is pertinent prior art, nor does it constitute any admission as to the contents or date of these publications or documents. All references cited herein are incorporated by reference to the same extent as if each individual publication and references were specifically and individually indicated to be incorporated by reference.

While the invention has been particularly shown and described with reference to a preferred embodiment thereof, it will be understood by those skilled in the art that various changes in form and details may be made therein without departing from the spirit and scope of the invention. As such, the invention is not defined by the discussion that appears above, but rather is defined by the claims that follow, the respective features recited in those claims, and by equivalents of such features.

What is claimed is:

1. A therapeutic composition comprising:
a. a plurality of quantum dots, each quantum dot encapsulated in an encapsulation compound transparent to a first wavelength spectrum, wherein the quantum dot is configured to absorb ambient light within the first wavelength range and emit light within a second wavelength range, where the second wavelength range is encompassed by the first wavelength range, wherein a first portion of the plurality of quantum dots are configured to emit light within the second wavelength range that is different from the light emitted by a second portion of the plurality of quantum dots.

2. A composition for use in treating a skin ailment, deficiency or non-aesthetic symptom, incorporating a light-conversion medium which receives ambient light of broad spectrum, and emits light at one or more peak wavelengths or ranges of wavelengths (λ1, λ2, λ3) having greater intensity than corresponding intensity in the received broad spectrum ambient light.

3. The composition of claim 2, wherein the one or more peak wavelengths or ranges of wavelengths λ1, λ2, λ3 are from about 200 nm to about 3000 nm, optionally in the UV, optionally from about 200 nm to about 400 nm, optionally in the UVA, optionally from about 310 nm to about 360 nm, optionally in the visible, optionally from about 400 nm to about 600 nm, optionally in the IR, optionally from about 600 nm to about 3000 nm, optionally in the NIR, optionally from about 810 nm to about 850 nm.

4. The composition of claim 2, wherein the light-conversion medium comprises a carrier medium and a light conversion element.

5. The composition of claim 4, wherein the particulate light-conversion medium is supported by a light-transmissive support medium.

6. The composition of claim 5, wherein the particulate light-conversion medium is molecularly bonded to the support medium.

7. The composition of claim 5, wherein the support medium is selected from a rigid, semi-rigid or flexible one of a plastic material, a polymeric material, or an organic material.

8. The composition of claim 5, wherein the support medium is a fluid material, selected from one of a gel, a cream, or a liquid.

9. The composition of claim 7, wherein the support material is hydrophilic.

10. The therapeutic composition of claim 1, wherein the therapeutic composition further includes at least one antihypertensive vasodilator compound.

11. The composition of claim 2, wherein the light conversion medium is encapsulated in one or more biocompatible polymers so as to prevent direct contact of the light conversion medium with the subject.

12. The composition of claim 5, wherein the support medium is a film.

13. The composition of 2, wherein the ailment is selected from one of rosacea; eczema, wrinkles, sun spots, freckles, UV damage or hair loss.

14. The therapeutic composition of claim 1, wherein the therapeutic composition is formulated as a gel, cream, or ointment.

15. The composition of claim 2, wherein the product includes one or more additional therapeutic compounds.

16. A light spectrum conversion device for absorbing and converting sunlight or other available ambient light comprising at least one an excitation layer configured to receive ambient light and output a modified light spectrum output that contains 50% or more of its output radiant in the 625-675 nm spectral range, wherein the excitation layer includes one or more quantum dots configured to absorb 50% of light incident thereupon in the 500-600 nm range.

17. The light spectrum device of claim 16, the further comprising: a semi-reflective layer disposed between an ambient light source and the excitation layer, wherein the semi-reflective layer is configured to transmit light in a 500-600 nm wavelength range and reflect light greater than 600 nm.

18. The light spectrum device of claim 16, wherein the light-conversion medium comprises at least a second excitation layer configured to emit 50% or more of its output radiant in the 800-850 nm range.

19. The light spectrum device of claim 16, wherein the excitation layer medium includes a plurality quantum dots selected from at least one of single shell, multi-shell, heavy metal and/or non-heavy metal quantum dots.

20. The light spectrum device of claim 19, wherein the plurality of quantum are silicone based quantum dots.
